(19) United States
(12) Patent Application Publication
(10) Pub. No.: US 2010/0036259 A1
(43) Pub. Date: Feb. 11, 2010

(54) Uterine Infrared Thermal Imaging Device

(76) Inventors: Paul DiCarlo, Middleboro, MA (US); Jon T. McIntyre, Newton, MA (US)

Correspondence Address: FAY KAPLUN & MARCIN, LLP, 150 BROADWAY, SUITE 702, NEW YORK, NY, 10038, US

(21) Appl. No.: 12/491,496

(22) Filed: Jun. 25, 2009

Related U.S. Application Data
(60) Provisional application No. 61/086,276, filed on Aug. 5, 2008.

(51) Int. Cl. A61B 6/00

(52) U.S. Cl. 600/474

(57) ABSTRACT

A device for locating target tissue within a living body, comprises an elongate member extending from a proximal end which, when in an operative position, remains outside the body to a distal end which, when in the operative position, extends into the body to a location proximate to a target portion of tissue, the elongate member defining a working channel extending therethrough to a distal opening in the distal end and a radiation delivery apparatus delivering infrared radiation to a target area distal of the distal end of the elongate member in combination with a radiation receiving apparatus at a distal end of the elongate member receiving radiation reflected from the target area.

### PRIORITY CLAIM

This application claims the priority to the U.S. Provisional Application Ser. No. 61/086,276, entitled “Uterine Infrared Thermal Imaging Device” filed on Aug. 5, 2008. The specification of the above-identified application is incorporated herewith by reference.

### BACKGROUND

Many medical procedures require access to the female pelvic region, including, for example, pelvic embolizations, pelvic occlusions, cervical biopsies, cystoscopies, endometrial ablations and various other endovaginal procedures. Visual access for these procedures is generally provided using an endoscope inserted into the pelvic region to relay an image to an external display. A physician then uses the image to identify specific region to be worked on (i.e., locating a fibroid, cyst, tumor, etc. for treatment; locating a vein/artery; etc.). However, visualizing treatment areas is often difficult as the field of vision may be occluded by blood flow, etc.

### SUMMARY OF THE INVENTION

The present invention relates to a device and method for the identification of veins, arteries and other blood vessels in a female pelvis. The present invention comprises an elongated infrared imaging device with a working channel extending therethrough, wherein insertion of the infrared imaging device in vivo provides an image which may be used to identify temperature variations within the pelvic region, vein/artery inflammations, tumors, etc. The present invention seeks to identify inflamed veins/arteries, tumors, fibroids, etc. by way of infrared imaging, allowing physician to locate designated subsurface treatment areas.

The present invention is directed to a device for locating target tissue within a living body, comprising an elongate member extending from a proximal end which, when in an operative position, remains outside the body to a distal end which, when in the operative position, extends into the body to a location proximate to a target portion of tissue, the elongate member defining a working channel extending therethrough to a distal opening in the distal end and a radiation delivery apparatus delivering infrared radiation to a target area distal of the distal end of the elongate member in combination with a radiation receiving apparatus at a distal end of the elongate member receiving radiation reflected from the target area.

The present invention is further directed to a method for locating subsurface target tissue comprising the steps of inserting into the body an elongate device including at a distal end thereof an infrared radiation delivery apparatus and a radiation receiving apparatus positioned to receive infrared radiation transmitted thereto from the radiation delivery apparatus after reflection from tissue and illuminating a target area of tissue adjacent to the distal end in combination with analyzing information from the radiation receiving apparatus to determine temperature levels of the tissue from which the radiation is reflected and identifying body structures beneath the tissue from which the radiation is reflected based on the determined temperature levels.

### BRIEF DESCRIPTION OF THE DRAWINGS

The accompanying drawings illustrate the present invention.

FIG. 1 shows a device according to the present invention shown in vivo in the female pelvis;

FIG. 2 shows an cut-through view of the device of an exemplary design of the present invention;

FIG. 3 shows an infrared image of normal arterial activity; and

FIG. 4 shows an infrared image of abnormal arterial activity.

### DETAILED DESCRIPTION

The present invention, which may be further understood with reference to the following description and the appended drawings, relates to a system and method for the identification of areas to be treated (e.g., inflamed veins or arteries, tumors, fibroids, cysts, etc.) in the female pelvic region. Although the exemplary embodiments of the present invention are described below with respect to particular procedures for the female pelvic region, the description is not meant to limit the application of the invention, which may be employed in a plurality of endovaginal and other procedures in this region. Furthermore, the present invention may be used for the identification of treatment areas in other parts of the body including, but not limited to, lumens such as the throat, biliary ducts, tear ducts, milk ducts, blood vessels, intestines, the colon and the rectum.

The present invention comprises an infrared imaging device with a working channel extending therethrough so that, when inserted in vivo, the device provides near infrared images to identify temperature variations within the pelvic region or other target region. Those skilled in the art will understand that, as vein/artery inflammation, tumors, etc. typically radiate a greater amount of thermal energy than surrounding structures, these near infrared images capture a heat scene, conceptualizing the temperatures and shapes of objects within its viewing field and facilitating identification of these higher heat radiating structures to identify target treatment areas even when the images provided by normal visualization techniques are inadequate.

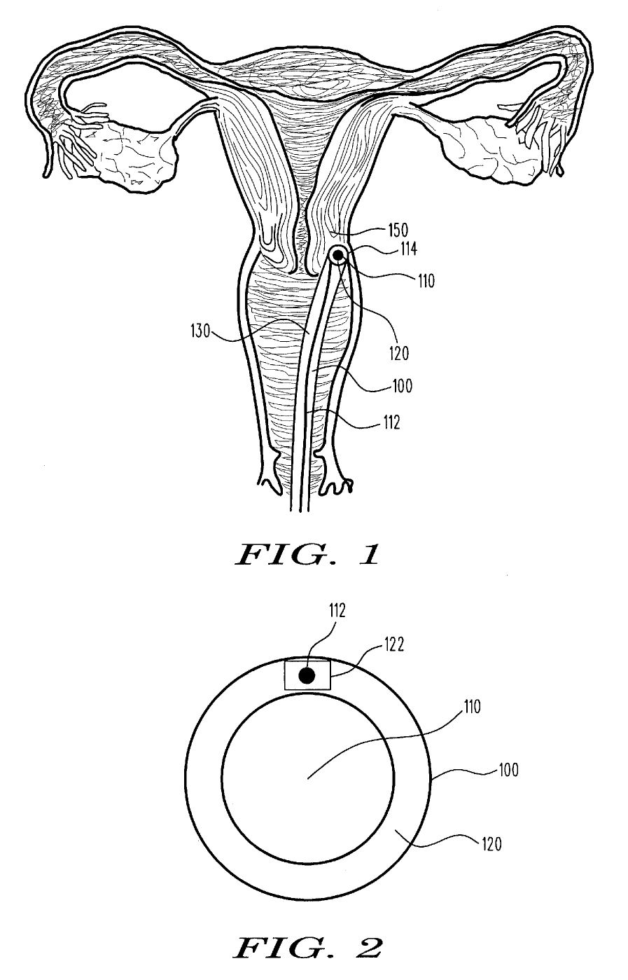

As shown in FIG. 1, a near-near infrared (“NIR”) device 100 according to an exemplary embodiment of the present invention comprises a flexible, elongated substantially tubular body 130 which, as would be understood by those skilled in the art preferably exhibits a column strength sufficient to enable to insertion of the NIR device 100 into the body (e.g., through a target lumen) without bunching. Furthermore, the elongated tubular body 130 of the NIR device 100 may comprise a length appropriate for a procedure being performed. In the embodiment of FIG. 1, a length of the elongated tubular body 130 is sufficient to enable its insertion to a target area of the female pelvic region, for example, adjacent to a vaginal formix 150. Those skilled in the art will understand that greater or lesser length may be employed in alternate embodiments and applications of the present invention depending on the intended use of the device.

The elongated tubular body 130 of the NIR device 100 is provided with a working channel 110 extending therethrough from a proximal end to a distal end 120. It is noted that the use of the term proximal hereinafter refers to a direction approaching a user of the device with the proximal most portion of the NIR device 100 remaining external to the body in an operative position. The term distal as used herein refers to a direction approaching a target site in a patient's body with the distal most portion of the device comprising an NIR imaging device 122. The NIR imaging device 122, which is located on the distal face of the NIR device 100 transmits captured images to a compatible external imaging device (not shown) either wirelessly or via a wire 112 extending through the body 130. As would be understood by those skilled in the art, the device 122 includes a source of infrared radiation as well as a sensor for receiving infrared radiation reflected from the target tissue. The sensor may be located at the distal end of the device or coupled to the distal end of the device via a fiber optic bundle or glass rod. The imaging components of the NIR device 100 may include any known infrared technology suitable for use within a living body. For example, the imaging device 122 may be substantially similar to endoscopic or laproscopic vision systems employing natural light except that the light source must produce the desired infrared wavelengths and the light receiving structure must be sensitive to these same wavelengths. Furthermore, as would be understood by those skilled in the art, the external imaging device may include, for example, a receiver to receive the image(s) from the NIR device 100 and a display screen.

In one potential application, the NIR device 100 may be deployed in the uterus to facilitate a utero-ovarian artery ligation procedure which, as those skilled in the art will understand, may be performed in cases of complications in pregnancy and childbirth. In an exemplary method according to the present invention, the NIR device 100 is inserted through the vaginal opening until the distal end 114 thereof is in a desired position adjacent to a target area at the vaginal formix 150 lateral to the cervical opening. This area is then imaged to locate the uterine artery to facilitate the performance of any known procedure involving the interruption of blood flow to the uterus.

The user of the device 100 initiates near infrared imaging by operating an actuator (not shown) on the proximal end of the device 100. A near infrared image is then transmitted to the external imaging device for display on a display screen with colors of the portions of the displayed image indicating the temperature of the various areas depicted. As would be understood by those skilled in the art, this temperature data may be interpreted to identify structures (e.g., arteries) and/or areas with abnormal vascular activity.

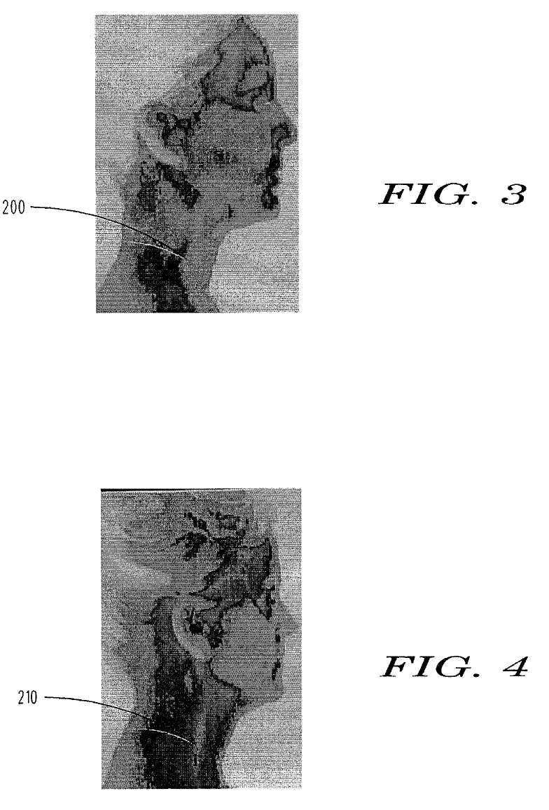

For example, as shown in the near infrared image of FIG. 3, the temperature distribution of an individual with a normally functioning carotid artery 200 renders the artery 200 substantially indistinguishable from surrounding tissue, etc. while the carotid artery 210 of the individual shown in FIG. 4 is clearly identifiable with this sharp temperature differentiation from surrounding tissue indicating inflammation. An inflamed carotid artery 210 is thereby easily located. However, even the normal carotid artery 200 may be identified in a similar manner by identifying the more subtle temperature differentials between it and the surrounding tissue. In this manner, images provided by the device 100 may be analyzed to locate either areas of unusual vascular activity (e.g., inflammation, tumor or cyst growth, etc.) or to locate healthy structures such as the uterine arteries. Accordingly, the NIR device 100 may enable the capturing of a near-infrared image of the uterine artery in vivo.

The imaging device 122 of the present invention possesses the functionality to capture a viewing field extending up to 1 inch radially outward from the distal end 120. Employing a viewing field of this size allows for a higher magnification per pixel of the captured near infrared image as compared to imaging capturing a wider viewing field. The higher pixel count per unit area of the image enables temperature differentials to be more finely differentiated. Accordingly, a user of the device 100 may detect more minute variations in temperature within the viewing field such as those required to identify healthy structures such as the uterine arteries. Furthermore, it is noted that the image captured and relayed to the external imaging device may be shown in real time to facilitate navigation of the NIR device 100 to the target area and/or between target areas.

In an alternate embodiment, the external imaging device may further be provided with controls that allow the physician to manipulate and browse through the near infrared image. For example, the external imaging device may comprise a zooming feature, allowing a physician to selectively zoom in or out of a selected area of the near infrared image. In an alternate embodiment, the external imaging device may be further provided with the functionality to patch multiple near infrared images together in order to provide a single image encompassing a greater viewing area while retaining the higher resolution obtained through the smaller viewing area. This feature may automatically, or, in the alternative, allow a physician to manually match and align overlapping areas of multiple near infrared images in order to create a larger image encompassing a greater length of the uterus. Furthermore, the external imaging device may also provide controls allowing the physician to alter color settings, wherein different colors may be associated with different temperatures, as those skilled in the art will understand and may include software which highlights areas showing a temperature distribution, differential or temperature value in a desired range to facilitate identifying target areas or structures.

For example, when a target uterine artery has been located, the user may perform any desired therapeutic or diagnostic procedure using this location to facilitate access to the structure. In an exemplary embodiment, the physician may insert a therapeutic device through the working channel 110 of the NIR device 100 to the target site on the vaginal formix 150 to penetrate the vaginal formix 150 and access the uterine artery, as those skilled in the art will understand. It is noted that the working channel 110 may be sized to properly accommodate a therapeutic device to be inserted therethrough such as, for example, a blunt dissection needle, a hemostatic clip or a device for injecting an embolic agent into the uterine artery. It is further noted that elongated tubular bodies 130 of various sizes may be provided to accommodate the required diameter of the working channel 110 for each of a variety of devices to be inserted therethrough while minimizing the diameter of the elongated tubular body 130 to reduce trauma and/or discomfort associated with its insertion.

To perform a utero-ovarian artery ligation procedure, as discussed previously, a device may be employed to place closure devices such as clips on the target blood vessel as close to the uterus as is possible. For example, any of the hemostatic clips described in U.S. Patent Application to Cohen, et al., entitled “Single Stage Mechanical Hemostasis Clipping Device,” filed May 3, 2007 and assigned Ser. No. 60/915,806 may be employed to perform the dissection and subsequent ligation of the uterine artery. In one embodiment, a suture clip (not shown), may be traversed through the working channel 110 of the NIR device 100 to a target site after a blunt dissection has been performed to expose the uterine artery. The suture clip may then be opened, placed over the artery and deployed in a known manner to stop blood flow through the uterine artery. A subsequent thermal image may then be taken to confirm that blood flow through the uterine artery has been substantially halted. Then, after blood flow has been halted for a desired time, the suture clip may be released to resume blood flow through the affected artery.

As those skilled in the art will understand, blunt dissection and ligation of the uterine arteries and/or other vessels has many applications, such as, for example, the cessation of hemorrhaging, shrinking of fibroids or other abnormal growths, etc. More specifically, by preventing the flow of blood to a region, the targeted region may be depraved of sustenance, to shrink or destroy the targeted region.

In one embodiment of the invention, a blunt needle may be employed, such as, for example the needle disclosed in U.S. application Ser. No. 11/446,946 entitled “Blunt Needles With Means for Locating and Occluding Vessels” to Sloan et al filed on Jun. 5, 2006, the entire contents of which are incorporated herein by reference. An exemplary method of use of this embodiment comprises advancing the NIR device 100 to a target site and capturing an near infrared image therein to locate a uterine artery. The blunt needle may then be advanced therethrough the working channel 110 wherein an incision may be formed in a side of a vaginal formix proximate to the uterine artery. The distal end of a blunt dissection needle may be advanced through the incision to a position adjacent to the uterine artery. An occlusion clip may then be deployed from the blunt dissection needle to clamp the uterine artery, wherein the occlusion clip is biased toward a clamping configuration. A subsequent near infrared image may be taken to confirm the accuracy of the occlusion prior to the retraction of the blunt needle from the body. After a predetermined period of time has elapsed, the uterine artery may then be released to restore blood flow therethrough. It is noted that the required period of time for the occlusion may vary based on the degree of severity of a fibroid, etc. and other factors. As those skilled in the art will understand, this time may be 6 hours or more, wherein the maximum time may be sufficient to necrose the fibroids but insufficient to permanently damage non-targeted tissue of the uterus. Alternatively, the clips may be left in place to permanently occlude flow through the arteries.

In yet another embodiment of the present invention, the suture device may comprise an ultrasound crystal located on a distal portion of the device. The ultrasound crystal may be used to further facilitate the detection and location of blood flow through the uterine artery or other vessel as would be understood by those skilled in the art.

Those skilled in the art will understand that the described exemplary embodiments of the present invention may be altered without departing from the spirit or scope of the invention. For example, the present invention may also be employed for imaging of and access to various vessels in a female pelvis or other hollow structures. Thus, it is to be understood that these embodiments have been described in an exemplary manner and are not intended to limit the scope of the invention which is intended to cover all modifications and variations of this invention that come within the scope of the appended claims and their equivalents.

What is claimed is:

1. A device for locating target tissue within a living body, comprising:
an elongate member extending from a proximal end which, when in an operative position, remains outside the body to a distal end which, when in the operative position, extends into the body to a location proximate to a target portion of tissue, the elongate member defining a working channel extending therethrough to a distal opening in the distal end;
a radiation delivery apparatus delivering infrared radiation to a target area distal of the distal end of the elongate member; and
a radiation receiving apparatus at a distal end of the elongate member receiving radiation reflected from the target area.

2. The device according to claim 1, wherein the radiation delivery apparatus includes one of a fiber optic bundle and a glass rod extending from the proximal end of the elongate member to the distal end thereof, the proximal end of the one of the fiber optic bundle and the glass rod being connectable to a source of infrared radiation.

3. The device according to claim 1, wherein the radiation delivery apparatus includes a source of infrared radiation mounted at the distal end of the elongate member.

4. The device according to claim 1, wherein the radiation receiving apparatus includes a structure sensitive to infrared radiation mounted at a distal end of the elongate member and converting infrared radiation incident thereupon into electrical impulses, the structure sensitive to infrared radiation being electrically coupled to a conductor extending through the elongate member to the proximal end thereof.

5. The device according to claim 1, wherein the radiation receiving apparatus includes one of a fiber optic bundle and a glass rod extending from the proximal end of the elongate member to the distal end thereof, the proximal end of the one of the fiber optic bundle and the glass rod being connectable to a structure sensitive to infrared radiation and converting infrared radiation incident thereupon into electrical impulses.

6. The device according to claim 1, further including an external display coupled to the radiation receiving apparatus.

7. The device according to claim 1, further comprising a processor receiving information from the radiation receiving apparatus and analyzing the information to determine temperature levels represented by different areas of an image field.

8. The device according to claim 1, wherein the radiation receiving apparatus transmits to the processor data enabling the processor to detect temperature differentials less than 0.08° C.

9. The device according to claim 7, wherein the elongate member includes a working channel extending therethrough to a distal opening permitting a user to insert therethrough tools for accessing tissue structures identified based on the temperature levels identified by the processor.

10. A method for locating subsurface target tissue comprising the steps of:
inserting into the body an elongate device including at a distal end thereof an infrared radiation delivery apparatus and a radiation receiving apparatus positioned to receive infrared radiation from the radiation delivery apparatus after reflection from tissue;
illuminating a target area of tissue adjacent to the distal end;
analyzing information from the radiation receiving apparatus to determine temperature levels of the tissue from which the radiation is reflected; and
identifying a subsurface body structure beneath the tissue from which the radiation is reflected based on the determined temperature levels.

11. The method of claim 10, further comprising inserting a therapeutic device through a working channel of the elongate device to perform a therapeutic procedure on the identified body structure.

12. The method of claim 11, wherein the identified body structure is a blood vessel and wherein the therapeutic device is adapted to cause hemostatis of the blood vessel.

13. The method of claim 12, wherein the therapeutic device includes one of a hemostatic clip and an embolic agent.

14. The method of claim 13, wherein the blood vessel is a uterine artery hemostasis of which is directed to the treatment of uterine fibroids.

15. The method of claim 14, wherein the elongate device is inserted to a target position adjacent a vaginal formix, wherein the uterine artery is accessed by advancing a blunt dissection needle through the vaginal formix to the uterine artery, hemostasis of the uterine artery being achieved by deploying one of the hemostatic clip and the embolic agent clip within the uterine artery.

16. The method of claim 15, further comprising the step of alleviating the hemostasis after a predetermined period of time has elapsed.

17. The method of claim 11, further comprising:
illuminating the target area of tissue after the therapeutic procedure has been completed; and
analyzing information from the radiation receiving apparatus.
